(19) United States
(12) Patent Application Publication
(10) Pub. No.: US 2016/0058818 A1
(43) Pub. Date: Mar. 3, 2016

(54) COMPOSITIONS AND METHODS FOR RELIEF FROM COLD SORES

(71) Applicants: Boris Gorinshteyn, Roswell, GA (US); F. Russell Denton, Lawrenceville, GA (US)

(72) Inventors: Boris Gorinshteyn, Roswell, GA (US); F. Russell Denton, Lawrenceville, GA (US)

(21) Appl. No.: 14/838,354

(22) Filed: Aug. 27, 2015

Related U.S. Application Data
(60) Provisional application No. 62/042,338, filed on Aug. 27, 2014.

(51) Int. Cl. A61K 36/752; A61K 31/4166; A61K 36/71; A61K 36/534; A61K 9/00; A61K 36/28

(52) U.S. Cl. CPC A61K 36/752; A61K 9/0014; A61K 36/28; A61K 36/71; A61K 36/534; A61K 31/4166

(57) ABSTRACT

The invention provides compositions and methods that relieve pain and reduce the duration of cold sore episodes to a period of at most 3 to 5 days. Specifically, the invention provides a combination of extracts of bitter orange (Citrus x aurantium), yarrow (Achillea millefolium) and at least one of goldenseal (Hydrastis canadensis)), barberry (Berberis vulgaris) and Chinese goldthread (Coptis chinensis); the invention may be used in the form of a cream, lipstick or other topical composition dosing format to treat and prevent cold sore episodes. In certain embodiments the compositions include further ingredients such as an analgesic natural composition from the mint genus (Mentha), a protective coat-forming substance, a substance for promoting general health of skin, a substance to modify the color of the product a substance to modify the flavor of the product, a preservative, and/or a substance to modify the texture, viscosity or mechanical strength of the composition.

### RELATED APPLICATIONS

This application derives priority from a U.S. provisional patent application having serial no. U.S. Ser. No. 62/042,338 filed Aug. 27, 2014 by the same inventors and having the same title.

### FIELD OF THE INVENTION

The invention provides compositions and methods for the treatment and prevention of cold sores, also known as fever blisters.

### BACKGROUND

Cold sores, also known as fever blisters, are endemic throughout the world. One large survey of young adults on six continents reported that 33.2% of males and 28% of females had an outbreak of the sores at least twice during the year before the study. The lifetime prevalence in the United States is estimated at 20% to 45% of the adult population. And antibodies against the virus can be found in about 80% of all adolescents. Although some individuals have cold sores only rarely, symptomatic subjects usually experience one to three attacks per year, and some subjects have as many as 12 or more recurrences per year.

The condition arises from a type of herpes simplex virus (HSV) that infects the lip, hence the name herpes labialis, also called herpes simplex labialis. Variations of HSV infection include: orolabial herpes (affecting mouth and lips); orofacial herpes (when the virus affects both mouth and face), and herpetic stomatitis (when the virus infects only the mouth). Infrequently the infection attacks the gums, alveolar ridge, hard palate, or back of the tongue.

When infecting the corner of the mouth, the infection (sometimes called angular herpes simplex) may be mistaken for a fungal infection, angular cheilitis. Herpes simplex infections arise from more than one strain; generally it is the HSV-1 strain, but HSV-2 is responsible for 10-15% of oral infections. The primary (i.e., initial) infection tends to begin when HSV contacts oral mucosal tissue or abraded skin of the mouth. In rare cases, newborn babies contract a severe case of neonatal herpes simplex when accidentally infected by family members or medical staff.

A typical outbreak of HSV-induced cold sores produces small blisters or sores on or around the mouth. Although these ordinarily heal in 2-3 weeks, the virus nevertheless continues to reside in a dormant state for the subject's lifetime in the facial sensory nerve ganglia (usually trigeminal). The dormant virus then reactivates periodically in symptomatic subjects, regenerating sores at the same area of the mouth or face. Over time the frequency and severity of sores tend to decrease for a given subject, however this may require several years.

The dormant stage is symptomless. The main symptom for oral infection is inflammation of the mucosa in the cheek and gums (acute herpetic gingivostomatitis) in the first 5 to 10 days of an outbreak. Other symptoms may include headache, nausea, dizziness, fever and sore throat, as well as painful ulcers that may be mistaken for canker sores. Primary HSV infection tends to manifest with severe sore throat. In adolescents it is commonly accompanied by lesions on the cheek and gums but this is less common in adults. Some subjects also have difficulty swallowing and may have swollen lymph nodes. HSV-1 infections are more likely than HSV-2 to produce recurrent oral infections.

The viral life cycle proceeds through eight stages. In the latent phase the subject is in remission and remains free of outbreak for weeks to months at a time. Next, the prodromal period lasts for hours to days, during which the infected skin reddens and has itch. This is conventionally regarded as the best time to begin medical treatment. The third stage is inflammation, when the virus begins reproducing and infecting cells at the nerve end over the course of a day; swelling and redness appear due to reactions by healthy cells. The following stage lasts about two days and manifests a pre-sore characterized by hard inflamed papules and itchy or painfully sensitive vesicles. This pre-sore phase eventually produces fluid-filled blisters that cluster on the lip, at its edge with skin, and sometimes on the nose, chin and cheeks. The fifth stage is a painful and highly contagious open lesion, where the vesicles have broken and merged into a weeping ulcer; this phase lasts about a day and may be accompanied by a fever and swollen lymph glands under the jaw.

The first five stages of accelerating infection are followed by three recovery stages. A four-day “crusting” stage forms yellowish or brown scabs from the exudates, that is now rich in immunoglobulins though it still contains virus; the sore is healing but still painful, and cracking of the scabs hurts even more. The seventh stage is healing, lasts about six days, and generates new skin beneath a series of shrinking scabs as the virus retreats into latency; irritation, itching and pain are still common. Finally, a post-scab stage in the last three days of healing is characterized by lingering redness, subject cell regeneration, and sometimes viral shedding.

Cold sores are commonly triggered by stress, menstruation, sunlight, sunburn, colds, fever, dehydration, or local skin trauma. They are also commonly triggered by dental and neural surgery, lip tattooing, and dermabrasion.

The unpredictability of onset and the transient nature of cold sores has hindered medical research for treatment. Common forms of treatment include use of antiviral drugs for up to 7 days, however these may not necessarily prevent symptomatic outbreaks, also they accelerate healing by less than two or three days and often by just a half-day. Illustrative antivirals used for this purpose include famcilovir and valaciclovir (each favored for a single-day, high dose pill), acyclovir and penciclovir (each typically used in lower doses for 5-7 days, and optionally combined with hydrocortisone). Oral acyclovir (800-1,600 mg daily) and valacyclovir (500 mg daily for 4 months) are said to prevent recurrent herpes labialis if taken prior to the onset of symptoms or exposure to triggers.

Other treatment alternatives are as follows. Anesthetic or non-treatment creams containing zinc oxide or zinc sulfate may provide pain relief for three to five days if used promptly upon appearance of symptoms, particularly if used simultaneously with antiviral drugs.

The amino acid lysine is under review as a treatment, but when administered to actual patients has failed to reproduce the benefits observed in test tube studies. Also, the saturated fatty alcohol docosanol is approved by the FDA for topical use in adults whose immune systems function properly; docosanol is comparable in efficacy to antiviral medicines.

Hence not only are cold sores ubiquitous, persistent and ineradicable in their epidemiology, but the best available treatments provide only limited relief from discomfort and the outbreak cycle. Thus there is an ongoing need for improved compositions to treat and prevent cold sores.

### SUMMARY OF THE INVENTION

The invention provides compositions that relieve pain and reduce the duration of cold sore episodes to a period of at most 3 to 5 days. Specifically, the invention provides a combination of extracts of: bitter orange (Citrus x aurantium); yarrow (Achillea millefolium); and at least one of goldenseal (Hydrastis canadensis), barberry (Berberis vulgaris) and Chinese goldthread (Coptis chinensis); the invention may be used in the form of a cream, lipstick or other dosing format to treat or prevent cold sore episodes. In certain embodiments the compositions include further ingredients such as an analgesic natural composition from the mint genus (Mentha), a protective coat-forming substance, a substance for promoting general health of skin, a substance to modify the color of the product, a substance to modify the flavor of the product, a preservative, and/or a substance to modify the texture, viscosity or mechanical strength of the composition.

In one illustrative embodiment the invention provides an aqueous topical composition comprising:
extract of bitter orange;
extract of yarrow;
an extract from at least one species selected from the group consisting of goldenseal, barberry and Chinese goldthread; and water;

wherein:
the extract of bitter orange is present in an amount that is within a range from 0.05% to 20% by weight of the topical composition;
the extract of yarrow extract is present in an amount that is within a range from 0.05% to 10% by weight of the topical composition;
the extract of at least one of goldenseal, barberry and Chinese goldthread is present in an amount that is within a range from 0.05% to 5% by weight of the topical composition;
the extracts are reconstituted in the water; and
the topical composition is a cream.

In another illustrative embodiment the invention provides a topical composition comprising:
extract of bitter orange;
extract of yarrow;
an extract from at least one species selected from the group consisting of goldenseal, barberry and Chinese goldthread;
at least one substance that is a wax; and
water;

wherein:
the extract of bitter orange is present in an amount that is within a range from 0.5% to 20% by weight of the topical composition;
the extract of yarrow is present in an amount that is within a range from 0.05% to 10% by weight of the topical composition;
the extract of at least one of goldenseal, barberry and Chinese goldthread is present in an amount that is within a range from 0.05% to 5% by weight of the topical composition;
the extracts are reconstituted in the water; and
the topical composition is a solid.

In a further illustrative embodiment the invention provides a method of treating a cold sore on a subject in need thereof, wherein the method comprises:
a) providing a composition that comprises water in which bitter orange extract, yarrow extract and an extract selected from at least one of a species selected from the group consisting of goldenseal, barberry and Chinese goldthread have been reconstituted in the water; and
b) rubbing the composition onto the surface of the cold sore.

### DETAILED DESCRIPTION OF THE INVENTION

The following definitions are provided to further clarify invention and its compositions, use thereof, and scope.

The term “treatment” as used herein refers to medical treatment; whether the treatment is therapeutic or prophylactic depends upon the context of the use.

The term “therapeutic” as used herein with respect to a wound refers to a treatment for the healing of that wound.

The terms “prophylactic” and “preventive” as used herein are synonymous and refer to a treatment to prevent formation of lesion.

The term “topical” as regards body tissues and application of a composition of the invention has its usual meaning in the fields of medicinal and cosmetic care.

The term “extract” as used herein has its normal and ordinary meaning in the art of medicinal herbal compositions, and includes but is not limited to: insoluble powders; soluble powders; miscible liquids; oils; concoctions; and extracts in a media format such as an alcohol, water or oil. The term “dried extract” as used herein refers to an extract from which extracting liquids such as water, alcohol and or another extracting liquid have been removed. Where the weight percentage of extract in a composition is recited herein, whether with or without the term “dried extract”, it means the percentage weight of plant-derived content relative to the overall composition, and does not imply that the extract exists in any particular form following formulation in the compositions.

In reference to an extract, the term “reconstituted in water” as used herein means that the extract has been mixed with water after being provided in an extracted form. As that term is used herein the water in which an extract is reconstituted may be substantially free of organic solvents, or alternatively may contain organic solvents.

The term “wax” as used herein has its usual and ordinary meaning in the field of cosmetic formulations. Nonlimiting illustrative examples of such substances include beeswax, emulsifying wax, jojoba esters, aliphatic waxes, and other cosmetic waxes.

The term “cream format” as used herein has its usual meaning in the fields of medicinal and cosmetic preparations.

The term “lipstick format” as used herein has its usual meaning in the fields of medicinal and cosmetic preparations.

The term “solid” as used herein has its usual meaning in the fields of chemistry and materials science, and includes but is not limited to solids that have a wax-like property.

The invention provides compositions that relieve pain and reduce the duration of cold sore episodes to a period of at most 3 to 5 days. Specifically, the invention provides a combination of extracts of bitter orange (Citrus x aurantium), yarrow (Achillea millefolium) and at least one of goldenseal (Hydrastis canadensis), barberry (Berberis vulgaris) and Chinese goldthread (Coptis chinensis); the invention may be used in the form of a cream, lipstick or other dosing format to treat or prevent cold sore episodes.

The invention further contemplates the use of botanical subspecies and varieties within the species taught here. Citrus x aurantium has subtaxa. Suitable subtaxa of Citrus x aurantium for use with the present invention include: subspecies amara, bigarade (Seville orange), myrtifolia, and daidai, but the invention is not so limited. Likewise, Achillea millefolium also has subtaxa. Suitable subtaxa of Achillea millefolium for use with the present invention include: subspecies millefolium and its varieties millefolium, borealis and rubra; subspecies chitralensis; and subspecies sudetica and its varieties alpicola, californica, occidentalis, pacifica and puberula, but the invention is not so limited.

Illustrative weight percent ranges for ingredients in compositions of the invention include the following. For extract of bitter orange illustrative ranges include: 0.050 to 20.0; 0.10 to 10.0; 0.20 to 9.0; 0.30 to 8.0; 0.40 to 7.0; 0.50 to 6.0; 0.60 to 5.0; 0.70 to 4.0; 0.80 to 3.0; 0.90 to 2.0;

or about 1.0 weight percent. For extract of yarrow illustrative ranges include: 0.025 to 10.0; 0.050 to 5.0; 0.10 to 4.5; 0.15 to 4.0; 0.20 to 3.5; 0.25 to 3.0; 0.30 to 2.5; 0.35 to 2.0; 0.40 to 1.5; 0.45 to 1.0; or about 0.50 weight percent. For total extract of one or any combination of goldenseal, barberry and Chinese goldthread, illustrative ranges include: 0.0125 to 5.0; 0.025 to 2.5; 0.050 to 2.25; 0.075 to 2.0; 0.10 to 1.75; 0.125 to 1.5; 0.15 to 1.25; 0.175 to 1.0; 0.20 to 0.75; 0.225 to 0.50; or about 0.25 weight percent. In a particularly useful embodiment the weight ratio of bitter orange:goldenseal: total of one or more of goldenseal, barberry and Chinese goldthread is 4:2:1, but the invention is not so limited.

Illustrative weight percent ranges for ingredients in compositions of the invention include the following. For a pain relieving agent illustrative ranges include: 1.0 to 20; 2.0 to 18.0; 3.0 to 16.0; 4.0 to 14.0; 5.0 to 12.0; 6.0 to 10.0; 7.0 to 8.0; or about 7.5 weight percent. Non-limiting illustrative examples of pain-relieving agents include 10 g benzocaine in 132 g cream, and 0.015 to 8.0 weight percent of a natural substance from the genus Mentha. For a protective coat-forming substance the proportion will depend on the format, for instance a lipstick-format dose may have a considerably higher proportion of wax-like substances than does a cream-format dose. Illustrative ranges for a cream format include: 1.0 to 15.0; 2.0 to 40.0; 4.0 to 36.0; 6.0 to 32.0; 8.0 to 28.0; 10.0 to 24.0; 12.0 to 20.0; 14.0 to 16.0; about 15.0 weight percent. A non-limiting illustrative protective coat-forming substance is a combination of 8.0 g cyclomethicone and 10.0 g emulsifying wax in 132 g cream.

In particular embodiments the invention further comprises an analgesic natural composition from the mint genus (Mentha) such as menthol, spearmint (Mentha spicata L.) oil, peppermint (Mentha piperita L.) oil or the like. In certain embodiments the analgesic is spearmint leaf oil, but the invention is not so limited.

In further embodiments the invention comprises a protective coat-forming substance. In certain embodiments the coat-forming substance is a polysiloxane such as dimethicone. In some embodiments the coat-forming substance is an aliphatic wax such as docosanol. In various embodiments the coat-forming substance is another hydrophobic substance, such as mineral oil, petroleum jelly or an emulsifying wax.

In various embodiments the invention comprises a substance to promote general health of skin. In some embodiments that substance is allantoin. Skin is defined herein, as in the field of medicine ordinarily, to include among other tissues the topical tissue of the lips. Illustrative examples of skin health promotion benefits include but are not limited to: moisturizing effects; keratolytic effects, hydration enhancement for the extracellular matrix; enhancement of desquamation of upper layers of dead skin cells; increasing the smoothness of the skin; promoting cell proliferation and wound healing: providing a soothing, anti-irritant, and skin protectant effect by forming complexes with irritant and sensitizing agents; modulating the inflammatory response; promoting fibroblast proliferation; promoting synthesis of the extracellular matrix; otherwise ameliorating the wound healing process; and reduction of irritation. An illustrative example of a substance for promotion of general skin health is allantoin. Illustrative ranges for incorporation of such substances is 0.5 to 10.0 weight percent of a composition of the invention, or about 5.0 weight percent, but the invention is not so limited. 1.0 to 20.0; 2.0 to 16.0; 3.0 to 12.0; 4.0 to 8.0; or about 5.0 weight percent. In some embodiments the invention comprises one or more additional substances to modify its color and/or flavor. Thus in certain embodiments a substance is provided that imparts a color that renders the composition more white, black, gray, brown, red, purple, orange, green, blue, yellow or some variant or intermediate of those colors. In various embodiments a substance is provided that conveys a flavor such as chocolate, mint, vanilla, orange, cherry, maple, cinnamon, amaretto, rum or another flavor. Non-limiting illustrative weight percent ranges for incorporation of color-modifying substances are: 0.0003 to 3.0; .001 to 1.0; and 0.03 to 0.3 weight percent. Non-limiting illustrative weight percent ranges for incorporation of flavoring substances are likewise: 0.0003 to 3.0; .001 to 1.0; and 0.03 to 0.3 weight percent.

In some embodiments compositions of the invention comprise an organic solvent. Non-limiting illustrative examples of organic solvents useful for the invention include methanol, ethanol, iso-propanol, n-propanol, tent-butanol, sec-butanol, n-butanol, dimethylsulfoxide, N-methylformamide, 1,2,3-propanetriol, 1,2-propanediol, and 1,2-ethanediol. Illustrative range for use of such solvents is 2.0 to 50.0; 4.0 to 40; 6.0 to 36.0; 8.0 to 32.0; 10.0 to 28.0; 12.0 to 24.0; 14.0 to 20.0; or about 16.0 weight percent. In certain embodiments the invention comprises one or more preservative substances. Nonlimiting illustrative examples include benzethonium chloride, benzalkonium chloride, hydrogen peroxide, and the like.

In various embodiments the invention comprises one or more additional substances. Nonlimiting illustrative examples include benzocaine for pain relief, witch hazel as an astringent, vitamin E and aloe in a cooling gel, phenylephrine as a vasoconstrictor, and the like.

In certain embodiments for stick-type formats, e.g., crayon-type or lipstick-type formats, substances may be included in the formulation to impart texture, relative mechanical strength and/or desired degree of viscosity. Illustrative nonlimiting embodiments include the following. A wax may be included, such as, e.g., beeswax, paraffin or carnauba wax. An oil or fat may be included, such as, e.g., lanolin oil, castor oil, olive oil or cocoa butter. An emollient may be included, such as, e.g., vitamin E or aloe vera. In some embodiments one or more of these substances is included in a cream-type formulation.

The following illustrate the preparation and use of compositions according to the invention, however the invention is not so limited.

### Formulation Example

A cream was formulated based on VersaProTM topical cream base from Medisca® (661 Route 3, Plattsburgh, N.Y. 12901, product no. 2529-0). The plant extracts were obtained from Naturex and corresponded to the respective standardization shown at www.naturex.com/data/classes/docu_fichier/docu_fichier_{—}7_{—}5.pdf. Approximately 100g VersaPro™ cream was blended with the following ingredients to form a visually homogeneous mixture: 2 g menthol, lg fruit peel extract of bitter orange (standardized at 3% Synephrine, 47% bioflavonoids), 0.5 g leaf and flower extract of yarrow (standardized at 4:1 strength), and 0.25 g root extract of goldenseal (standardized at 5% hydrastine). The following additional ingredients were also added: lOg benzocaine; 8 g cyclomethicone; and lOg emulsifying wax. The resulting cream had a dark greenish brown vegetative-like hue.

### Use Example

Subjects rubbed the cream of the formulation example gently onto cold sores four times per day beginning within 72 hours after the cold sores appeared. Despite its dark color in bulk form, upon application the cream blended with the skin tone in a seemingly clear and colorless way. In preliminary results the fever blisters healed within at most 3 to 5 days after the onset of application, whereas on average for these subjects two weeks was typical for the sores to run their course and heal without treatment. Their tingling, pain, burning, itching and other discomfort from cold sores was relieved within minutes of the cream's application, and their inflammation was virtually eliminated within one day after the initial application. Re-infection was not observed at the same or nearby topical sites within the duration of the study, which took place over a two month period.

Further clinical findings are that compositions of the invention resolve emerging fever blisters within one day if applied promptly at the outset of the manifestation. Moreover, even for initial application at a later stage the composition is found to have heightened efficacy when it has been used to treat a previous break-out; this effect is similar to that of acyclovir and is believed to arise from slowing the ability of the virus to mutate, thereby avoiding development of resistance to the body's existing pool of antibodies, however the invention is not limited by this hypothesis.

The embodiments of the invention as described herein are merely illustrative and are not exclusive. Numerous additions, variations, derivations, permutations, equivalents, combinations and modifications of the above-described composition and methods will be apparent to persons of ordinary skill in the relevant arts. The invention as described herein contemplates the use of those alternative embodiments without limitation.

1) An aqueous topical composition comprising:
extract of bitter orange;
extract of yarrow;
an extract selected from at least one species selected from the group consisting of goldenseal, barberry and Chinese goldthread; and
water;
wherein:
the extract of bitter orange is present in an amount that is within a range from 0.05% to 20% by weight of the topical composition;
the extract of yarrow extract is present in an amount that is within a range from 0.05% to 10% by weight of the topical composition;
the extract of at least one of goldenseal, barberry and Chinese goldthread is present in an amount that is within a range from 0.05% to 5% by weight of the topical composition;
the extracts are reconstituted in the water; and
the topical composition is a cream.

2) The topical composition of claim 1, further comprising a natural substance from the genus Mentha.

3) The topical composition of claim 2, wherein:
a) the natural substance from the genus Mentha is selected from the group consisting of menthol, spearmint oil or peppermint oil; and
b) the natural substance from the genus Mentha is present in an amount that is within a range of 0.015% to 8% by weight relative to the topical composition.

4) The topical composition of claim 1, further comprising a coat-forming substance.

5) The topical composition of claim 4, wherein the coat-forming substance is present in an amount that is within a range from 1% to 15% by weight.

6) The topical composition of claim 1, further comprising a pharmaceutically acceptable substance selected from the group consisting of flavoring compounds and food coloring compounds.

7) The topical composition of claim 6, wherein the concentration of a pharmaceutically acceptable substance selected from the group consisting of flavoring compounds and food coloring compounds is present in an amount that is within a range from 0.0003% to 3.0% of the topical composition by weight.

8) The topical composition of claim 6, wherein the pharmaceutically acceptable substance is a flavoring composition that imparts a flavor selected from the group consisting of chocolate, mint, vanilla, orange, cherry, maple, cinnamon, amaretto and rum.

9) The topical composition of claim 1, further comprising an organic solvent selected from the group consisting of methanol, ethanol, iso-propanol, n-propanol, tent-butanol, sec-butanol, n-butanol, dimethylsulfoxide, N-methylformamide, 1,2,3-propanetriol, 1,2-propanediol, and 1,2-ethanediol.

10) The topical composition of claim 9, wherein the concentration of the organic solvent ranges from 2% to 50% of the aggregate volume of liquid components of the topical composition, and wherein the concentration of water ranges from 50% to 98% of the aggregate volume of the liquid components of the topical composition.

11) The topical composition of claim 1, further comprising a substance for promoting general health of skin.

12) The topical composition of claim 11, wherein the substance for promoting general health of skin is allantoin.

13) A topical composition comprising:
extract of bitter orange;
extract of yarrow;
an extract selected from at least one species selected from the group consisting of goldenseal, barberry and Chinese goldthread;
at least one substance that is a wax; and
water;
wherein:
the extract of bitter orange is present in an amount that is within a range from 0.5% to 20% by weight of the topical composition;
the extract of yarrow is present in an amount that is within a range from 0.05% to 10% by weight of the topical composition;
the extract of at least one of goldenseal, barberry and Chinese goldthread is present in an amount that is within a range from 0.05% to 5% by weight of the topical composition;
the extracts are reconstituted in the water; and
the topical composition is a solid.

14) The topical composition of claim 13, wherein the wax is selected from the group consisting of beeswax, paraffin and carnauba wax.

15) The topical composition of claim 13, further comprising a natural substance from the genus Mentha.

16) The topical composition of claim 15, wherein:
a) the natural substance from the genus Mentha is selected from the group consisting of menthol, spearmint oil or peppermint oil; and
b) the natural substance from the genus Mentha is present in an amount that is within a range of 0.015% to 8% by weight relative to the topical composition.

17) The topical composition of claim 13, further comprising a coat-forming substance.

18) The topical composition of claim 17, wherein the concentration of a coat-forming substance ranges from 1% to 15% by weight.

19) The topical composition of claim 13, further comprising a pharmaceutically acceptable substance selected from the group consisting of flavoring compounds and food coloring compounds.

20) The topical composition of claim 19, wherein the pharmaceutically acceptable substance is present in an amount that is within in a range from 0.0003% to 3.0% of the composition by weight relative to the topical composition.

21) The topical composition of claim 19, wherein pharmaceutically acceptable substance is a flavoring composition that imparts a flavor selected from the group consisting of chocolate, mint, vanilla, orange, cherry, maple, cinnamon, amaretto and rum.

22) The topical composition of claim 13, further comprising an organic solvent that is selected from the group consisting of methanol, ethanol, iso-propanol, n-propanol, tert-butanol, sec-butanol, n-butanol, dimethylsulfoxide, N-methylformamide, 1,2,3-propanetriol, 1,2-propanediol, and 1,2-ethanediol.

23) The topical composition of claim 13, further comprising a substance for promoting general health of skin.

24) The topical composition of claim 23, wherein the substance for promoting general health of skin is allantoin.

25) A method of treating a cold sore on a subject in need thereof, wherein the method comprises:
a) providing a composition that comprises water in which bitter orange extract, yarrow extract and an extract selected from at least one of a species selected from the group consisting of goldenseal, barberry and Chinese goldthread have been reconstituted in the water; and
b) rubbing the composition onto the surface of the cold sore.

26) The method of claim 25, wherein the treatment is applied until the cold sore is no longer visible, and during that time is applied with a frequency that is selected from the group consisting of from 2 to 12 times daily.

27) The method of claim 25, wherein the composition is in a cream format.

28) The method of claim 25, wherein the composition is in a lipstick format.

29) The method of claim 25, wherein the extracts are present in the composition in the following amounts, based on the dry weight of the extract before reconstitution:
the bitter orange extract is present in an amount that is within a range from 0.05% to 20% by weight of the topical composition;
the yarrow extract is present in an amount that is within a range from 0.05% to 10% by weight of the topical composition; and
the extract from at least one species selected from the group consisting of goldenseal, barberry and Chinese goldthread is present in an amount that is within a range from 0.05% to 5% by weight of the topical composition.

30) The method of claim 25, wherein the topical composition further comprises a natural substance from the genus Mentha, wherein:
a) the natural substance from the genus Mentha is selected from the group consisting of menthol, spearmint oil or peppermint oil; and
b) the natural substance from the genus Mentha is present in an amount that is within a range of 0.015% to 8% by weight relative to the topical composition.

31) The method of claim 25, wherein the topical composition further comprises a coat-forming substance in an amount that is within a range from 1% to 15% by weight relative to the topical composition.

32) The method of claim 25 wherein the topical composition further comprises a pharmaceutically acceptable flavoring composition wherein:
a) the flavoring composition is present in an amount that is within a range from 0.0003% to 3.0% of the topical composition by weight; and
b) The flavoring composition imparts a flavor that is selected from the group consisting of chocolate, mint, vanilla, orange, cherry, maple, cinnamon, amaretto and rum.

33) The method of claim 25, wherein the topical composition further comprises an organic solvent selected from the group consisting of methanol, ethanol, iso-propanol, n-propanol, tent-butanol, sec-butanol, n-butanol, dimethylsulfoxide, N-methylformamide, 1,2,3-propanetriol, 1,2-propanediol, and 1,2-ethanediol.

34) The topical composition of claim 25, wherein the topical composition further comprises a wax that is selected from the group consisting of beeswax, paraffin and carnauba wax.

35) The topical composition of claim 25, further comprising a substance for promoting general health of skin.

36) The topical composition of claim 35, wherein the substance for promoting general health of skin is allantoin.
